(19) United States
(12) Patent Application Publication
(10) Pub. No.: US 2017/0234805 A1
(43) Pub. Date: Aug. 17, 2017

(54) MOBILE GEMSTONE IDENTIFICATION

(71) Applicant: SAHAJANAND TECHNOLOGIES PRIVATE LIMITED, Surat (IN)

(72) Inventors: Balkrishna Mahajan, Pune (IN); Nishma Pandit, Pune (IN); Chetan Patel, Surat (IN); Rahul Gaywala, Surat (IN); Bhargav Kotadia, Bethesda, MD (US)

(73) Assignee: SAHAJANAND TECHNOLOGIES PRIVATE LIMITED, Surat (IN)

(21) Appl. No.: 15/507,161

(22) PCT Filed: Aug. 26, 2015
(86) PCT No.: PCT/IB2015/056464

§ 371 Date: Feb. 27, 2017

(30) Foreign Application Priority Data
Aug. 27, 2014 (IN) 2751/MUM/2014

(51) Int. Cl. G01N 21/87; G01N 33/38

(52) U.S. Cl. CPC G01N 21/87; G01N 33/381

(57) ABSTRACT

Systems and methods for mobile gemstone identification are described herein. According to an embodiment, a mobile gemstone identification system (100) includes a gemstone holder (104, 202, 302, 402) to hold a gemstone (102) and includes an optoelectronic assembly (106). The optoelectronic assembly (106) includes an illumination device (108) to illuminate the gemstone (102) by causing radiations to be incident on the gemstone (102). Further, the optoelectronic assembly (106) further includes a screen (110) to form a pattern indicative of an optical response of the gemstone (102), in response to the radiations being incident on the gemstone (102). According to an aspect, the screen (110) is formed as having a plurality of regions (114, 116) having different masses, and the pattern is to be formed substantially on a region (114) having greater mass than other regions (116).

### TECHNICAL FIELD

The present subject matter relates, in general, to gemstone identification and, particularly but not exclusively, to identification of a gemstone using optics.

### BACKGROUND

The value grade, referred to as quality, of a gemstone is generally assessed in accordance with weight of the gemstone, cut of the gemstone, clarity of the gemstone, color of the gemstone, and finish of the gemstone. For example, for assessing the quality of a diamond, the amount and type of impurities in the gemstone are determined at an atomic level within the crystal lattice of carbon atoms. Owing to the enormous value of quality gemstones, such as diamonds, counterfeit gemstones are produced, or the quality of the less-valued gemstones is enhanced artificially. In either case, the size and sophisticated structure of the gemstones make it difficult to differentiate among gemstones with the naked eye. Therefore, conventionally, techniques are developed for identifying original gemstones from counterfeit or less-valued gemstones. Generally, such conventional techniques deploy optics for the identification of gemstones.

### BRIEF DESCRIPTION OF DRAWINGS

The detailed description is described with reference to the accompanying figures. In the figures, the left-most digit(s) of a reference number identifies the figure in which the reference number first appears. The same numbers are used throughout the drawings to reference like features and components.

FIG. 1A and FIG. 1B illustrate a mobile gemstone identification device for identifying a gemstone, in accordance with an embodiment of the present subject matter.

FIG. 1C illustrates a screen of the mobile gemstone identification system, according to an embodiment of the present subject matter.

FIG. 1D illustrates the screen of the mobile gemstone identification system, according to another embodiment of the present subject matter.

FIG. 2 illustrates the mobile gemstone identification system, in accordance with another embodiment of the present subject matter.

FIG. 3 illustrates the mobile gemstone identification system, in accordance with one other embodiment of the present subject matter.

FIG. 4 illustrates the mobile gemstone identification system, in accordance with yet another embodiment of the present subject matter.

### DETAILED DESCRIPTION

The present subject matter relates to identification of gemstones using optics, in accordance with an aspect of the present subject matter. A gemstone is a cut of a mineral and can be processed and polished for use in jewellery, other adornments, and even decorative items. Examples of gemstones can include diamonds, rubies, and sapphires. As gemstones are precious and costly, they are usually assessed for authenticity before being traded. Conventional techniques for identifying the gemstones involve illuminating the gemstone and capturing a reflection pattern generated by the gemstone. Each gemstone, due to its inherent properties, produces a unique reflection pattern and the pattern is used for identifying the gemstone, for example, to verify authenticity of the gemstone.

The set-up for identifying the gemstone includes a holder for holding the gemstone, an illuminating device to illuminate the gemstone, a screen for the reflection pattern of the gemstone to form, and a camera for capturing the reflection pattern. For the reflection pattern to be of a discernible size, the gemstone and the camera have to be positioned considerably far from the screen. Therefore, the overall size of the set-up is considerably large. For instance, the size of the set-up can be around 12 inches×12 inches×12 inches, measured in terms of length×width×height. In turn, the large size of the setup allows it for being used in stationary applications and renders it unusable for mobile applications.

The present subject matter relates to a mobile gemstone identification system and a method for mobile gemstone identification, in accordance with an aspect of the present subject matter. The mobile gemstone identification system, in accordance with the present subject matter, is a compact and portable unit and, therefore, can be used for mobile applications, for example, in hand-held devices.

In an embodiment, the mobile gemstone identification system includes a gemstone holder for holding the gemstone and an optoelectronic assembly for capturing a unique pattern of the gemstone during operation. As will be understood, the gemstone can include a bare gemstone or a gemstone fitted in an ornament, such as jewellery. The optoelectronic assembly can include an illumination device which incidents radiations on the gemstone and a screen for forming a pattern indicative of an optical response of the gemstone, in response to the radiations incident on the gemstone. In one example, the illumination device can be a laser source. Further, according to an aspect of the present subject matter, the screen can be formed as having a plurality of regions, the plurality of regions having different masses. In an example, the screen can be formed as having a first region and a second region, the first region having greater mass than the second region. In one case, the first region can have greater density than the second region. In another case, the first region can have a different cross-sectional thickness than the second region. For instance, the first region can have a greater cross-sectional thickness than the second region. The pattern associated with the gemstone can be formed substantially on the region having greater mass, during operation. The pattern so formed on the screen can be captured for identification of the gemstone, say by comparing with previously stored patterns.

The greater mass of one region of the screen can provide for forming a substantially clear and distinctly visible image on the screen, say even when the distance between the gemstone and the screen is substantially less. Accordingly, the overall size of the mobile gemstone identification system can be considerably less as compared to the gemstone identification units used conventionally, without affecting the quality of the image formed.

For the purpose of providing greater mass in one region than other regions of the screen, according to an embodiment, the screen can be formed as having a curvature at least on the surface of the screen facing the gemstone. In an example, the screen can have a biconvex cross section, a plano-convex cross section, biconcave cross section, or a plano-concave cross section. As would be understood, the cross section of the screen refers to a section of the screen along a plane substantially perpendicular to the screen. In another embodiment, the screen can be formed as having a layered structure to form a structure of the screen having varying thickness. Accordingly, in the above mentioned embodiments, the screen can have different regions of varying mass, for example, considering that the density of the material of the screen is substantially consistent throughout the volume of the screen.

Further, in an implementation, the region having greater mass can have a darker shade than the other regions, say on a surface of the screen facing the gemstone. Such a provision facilitates in attenuating the pattern of the gemstone when formed on the screen so that a clear image of the pattern is captured for identification.

In addition, for capturing the pattern of the gemstone formed on the screen, the optoelectronic assembly can include an image capturing device, say a camera. In another example, the mobile gemstone identification system can be used in combination with a separate image capturing device. In such a case, the gemstone can be fitted into the gemstone holder and the separate image capturing device can be used for capturing the image. The captured image can further be used for the identification of the gemstone. Such a provision enhances the portability of the mobile gemstone identification system.

Further, in order to make the mobile gemstone identification system further compact, the gemstone holder can be so positioned with respect to the illumination device that the two are non-linearly placed, i.e., one is not in a straight line with respect to the other. For example, the illumination device can be positioned to incident the radiations in a direction substantially perpendicular to the gemstone. In such a case, in an implementation, the optoelectronic assembly can include an optical device, such as a prism, for directing the radiations from the illumination device onto the gemstone.

In addition, the mobile gemstone identification system can be adapted to achieve successive identification of a plurality of gemstones. Accordingly in one implementation, the mobile gemstone identification system can include a conveyor belt having a plurality of gemstone holders provided thereon and each gemstone holder can be used for grasping the gemstone. The conveyor belt can be provided with a drive through an actuator, in turn driven by a processing unit, to regulate the positioning of the gemstones for capturing the pattern.

In another implementation, the mobile gemstone identification system can include a multi-holder plate having a plurality of gemstone holders and an actuator assembly coupled to the multi-holder plate and/or the optoelectronic assembly for achieving a relative motion between the gemstones and the optoelectronic assembly for positioning the optoelectronic assembly with respect to the gemstone. In another case, an actuator assembly can be individually coupled to each gemstone holder on the multi-holder plate to individually regulate the movement of the gemstone holder with respect to the optoelectronic assembly.

In addition, the mobile gemstone identification system is adapted to couple to a global database having various certificates for the identified, authenticate gemstones, against which the gemstone provided at the mobile gemstone identification system can be checked and verified or identified. Additionally, in case the pattern of the gemstone does not match with any existing pattern in the database, the pattern of the gemstone can be updated in the global database, say after it has been authenticated.

In addition, the present subject matter relates to the method for mobile gemstone identification. In an implementation, the method can include securing a gemstone in a gemstone holder, illuminating the gemstone, and capturing the pattern indicative of the optical response of the gemstone formed on a screen, in response to the radiations being incident on the gemstone. As explained above, the screen is formed as having a curvature on a surface facing the gemstone and the pattern of the gemstone being formed on the curved surface.

These and other advantages of the present subject matter would be described in greater detail in conjunction with the following figures. While aspects of described systems and methods for mobile identification of gemstones can be implemented in any number of different computing systems, environments, and/or configurations, the embodiments are described in the context of the following device(s).

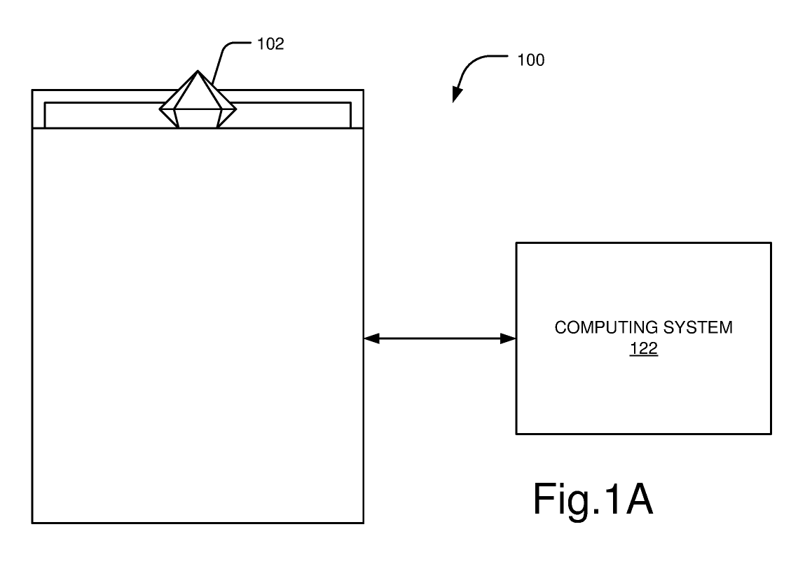

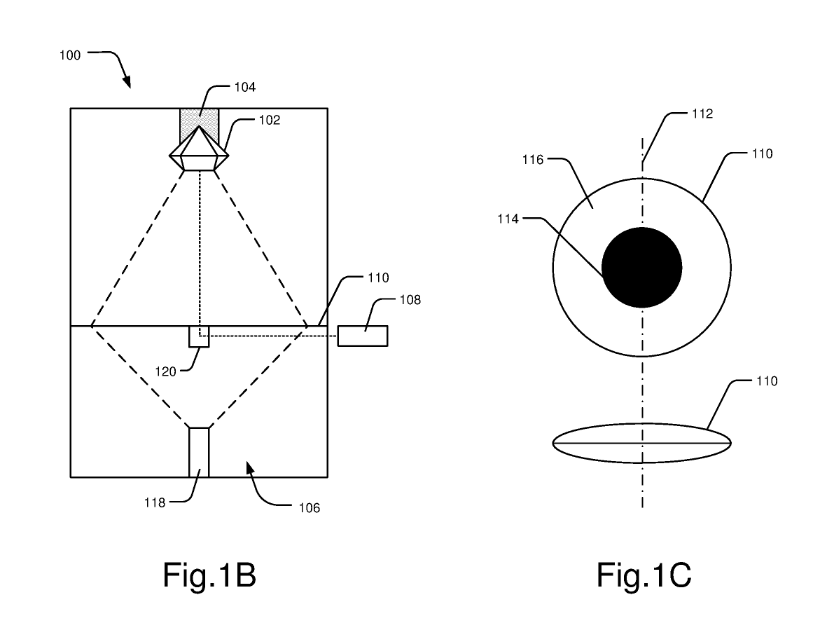

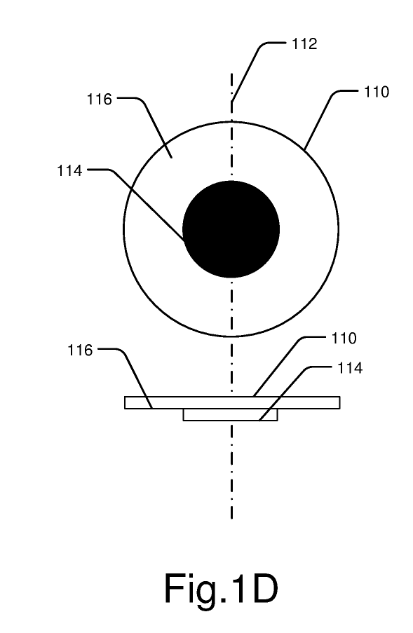

FIG. 1A and FIG. 1B illustrate a mobile gemstone identification system 100 for identifying a gemstone 102, in accordance with an embodiment of the present subject matter. While FIG. 1A illustrates a front view of the mobile gemstone identification system 100, FIG. 1B illustrates a sectional view of the mobile gemstone identification system 100 showing various components of the mobile gemstone identification system 100. For the sake of brevity, the description with respect to FIG. 1A and FIG. 1B is provided in conjunction.

According to an embodiment of the present subject matter, the mobile gemstone identification system 100, hereinafter referred to as the system 100 includes a gemstone holder 104 for holding the gemstone 102 and an optoelectronic assembly 106 for capturing a unique pattern of the gemstone 102 during operation. As will be understood, the gemstone 102 can include a bare gemstone or a gemstone fitted in an ornament, such as jewellery. The optoelectronic assembly 106 can include an illumination device 108 which incidents radiations on the gemstone 102 during operation and a screen 110 for forming a pattern indicative of an optical response of the gemstone 102, in response to the radiations incident on the gemstone 102. In one example, the illumination device 108 can be a laser source.

Further, according to an aspect of the present subject matter, the screen 110 can be formed as having a plurality of regions having different masses. According to an aspect, the pattern associated with the gemstone 102 is formed substantially on the region of the screen 110 having greater mass. The pattern so formed on the screen 110 can be captured for identification of the gemstone 102, say by comparing with previously stored patterns. The greater mass in different regions of the screen can provide for forming a substantially clear and distinctly visible image on the screen 110, say even when the distance between the gemstone and the screen is substantially less. In an example, the distance between the gemstone 102 and the screen 110 can vary depending on the optical arrangements. For instance, the distance between the gemstone 102 and the screen 110 can be about 4 inches. Accordingly, the provision of such a structure of the screen 110 can facilitate in reducing the overall size of the system 100 in comparison to size of the conventional gemstone identification units, without affecting the quality of the image formed. According to an aspect, the greater mass of the screen 100 in different regions can be achieved in various ways. For instance, different material or thickness in different regions of the screen 110 can be achieved for the purpose of varying the mass of the screen 100 in different regions.

According an implementation, the screen 110 can be formed as having a first region 114 and a second region 116, the first region 114 having greater mass than the second region 116. In an example, the first region 114 can have greater density than the second region 116. In such a case, the first region 114 and the second 116 of the screen 110 can be formed of different materials. In another example, the first region 114 can have different cross-sectional thickness than the second region 116. For instance, the first region 114 can have a greater cross sectional thickness than the second region 116. In yet another example, the first region 114 can have greater density as well as greater thickness than the second region 116. FIG. 1C illustrates the screen 110, in accordance with an embodiment of the present subject matter. For the purpose of providing greater mass in the first region 114 than the second region 116, according to said embodiment, the screen 110 can be formed as having a curvature on at least the surface facing the gemstone. As shown in FIG. 1C, in an example, the screen 110 can have a biconvex cross section. However, in other cases, the screen 110 can have a plano-convex cross section, biconcave cross section, or a plano-concave cross section. As would be understood, the cross section of the screen 110 refers to a section of the screen 110 along a plane 112 substantially perpendicular to the screen 110.

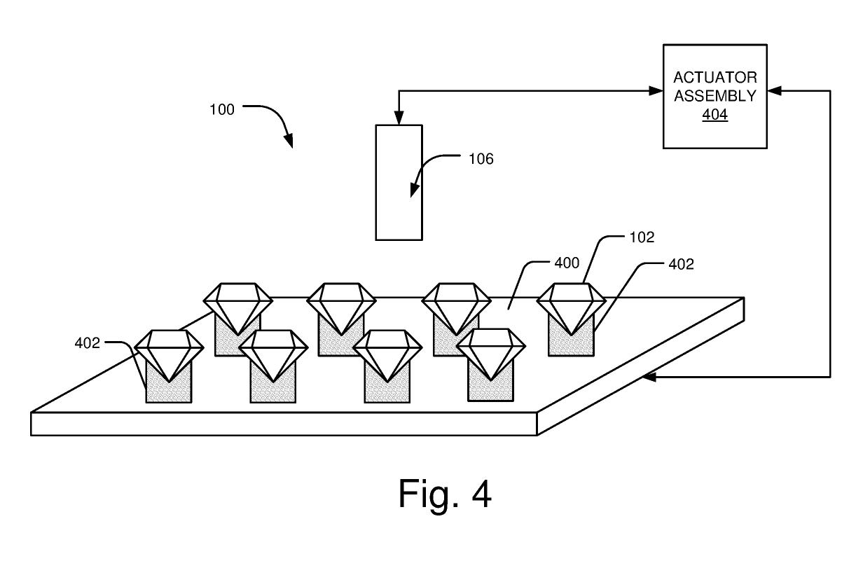

Further, FIG. 1D illustrates the screen 110 of the mobile gemstone identification system 100, in accordance with another embodiment of the present subject matter. In said embodiment, the screen 110 can be formed as having a layered structure to form a structure of the screen 110 having varying thickness. The layered structure of the screen 110 according to the present embodiment as shown in FIG. 4 depicts the first region 114 having a greater thickness than the second region 116.

In the above mentioned embodiments shown in FIG. 1C and FIG. 1D, the screen 110 can have different regions of varying mass, for example, considering that the density of the material of the screen 110 is substantially consistent throughout the volume of the screen 110.

In addition, in accordance with an aspect of the present subject matter, the plurality of regions 114, 116 of the screen can differentiate in their shades. For example, the region on which the pattern of the gemstone is to be formed can have a darker shade than the other regions, on a surface facing the gemstone. In the examples shown in FIG. 1C and FIG. 1D, in which the screen 110 is formed as having the first region 114 and the second region 116, the first region 114 can have a darker shade than the second region 116, and the pattern of the gemstone 102 can be formed substantially on the first region 114, i.e., the darker region, during operation. Providing such differently shaded regions on the screen allows attenuation of the pattern of the gemstone 102 when formed on the screen 100 so that a clear image of the pattern is captured for identification.

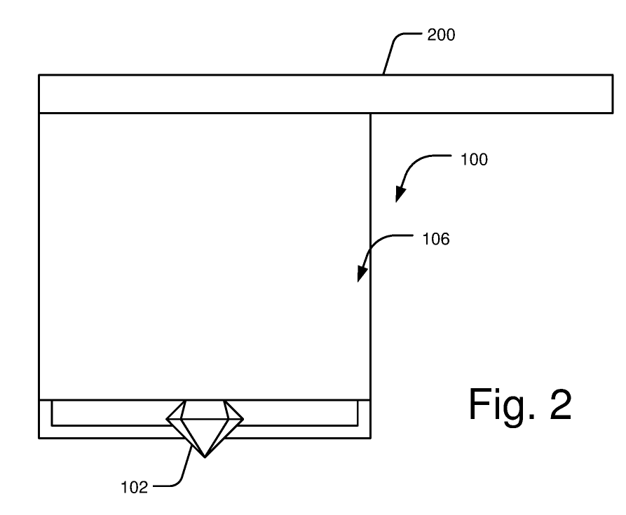

In addition, for capturing the pattern of the gemstone 102 formed on the screen, the optoelectronic assembly 106 can include an image capturing device 118, say a camera. In an embodiment, the image capturing device 118 can be integrated with the system 100. In another embodiment, the system 100 can be used in combination with a separate or non-integrated image capturing device 118. The latter embodiment is discussed with respect to FIG. 2.

The system 100, in accordance with the present subject matter is a compact and portable unit and, therefore, can be used for mobile applications. For instance, the system 100 can be implemented as a hand-held device for mobility of use of the system 110. In an example, the system 100 can have the dimensions of 4 inches×4 inches×2 inches, measured in terms of length×width×height.

In an implementation, the gemstone holder 104 can be so positioned with respect to the illumination device 108 that the two are linearly placed, i.e., the two are in a straight line. Further, in order to make the system 100 further compact, in another implementation, the gemstone holder 104 can be so positioned with respect to the illumination device 108 that the two are non-linearly placed, i.e., the two are not in a straight line. For example, the illumination device 108 can be positioned to incident the radiations in a direction substantially perpendicular to the gemstone holder 104 holding the gemstone 102. In such a case, in an implementation, the optoelectronic assembly 106 can include an optical device 120, such as a prism, for directing the radiations from the illumination device 108 onto the gemstone 102.

In addition, the system 100 can be adapted to couple to a global database (not shown) having a various certificates for the identified, authenticate gemstones, against which the gemstone 102 provided at the system 100 can be checked and verified or identified. In an example, the system 100 can be coupled to the global database through a computing system 122. For instance, the computing system 122 can be implemented as laptop computer, a desktop computer, a notebook, a tablet, a smart phone, a workstation, a mainframe computer, a server, and the like. Additionally, in case the pattern of the gemstone 102 does not match with any existing pattern in the global database, the pattern of the gemstone 102 can be updated in the global database, say after it has been authenticated.

FIG. 2 illustrates the system 100, in accordance with another embodiment of the present subject matter. As mentioned above, a separate image capturing device 200 can be used with the system 100. In such a case, the gemstone 102 can be fitted into the gemstone holder 104 and the separate image capturing device 200 can be used for capturing the image. In an example, the image capturing device 200 can be a smart phone camera or any other type of digital camera. Such a provision can further enhance the portability of the system 100.

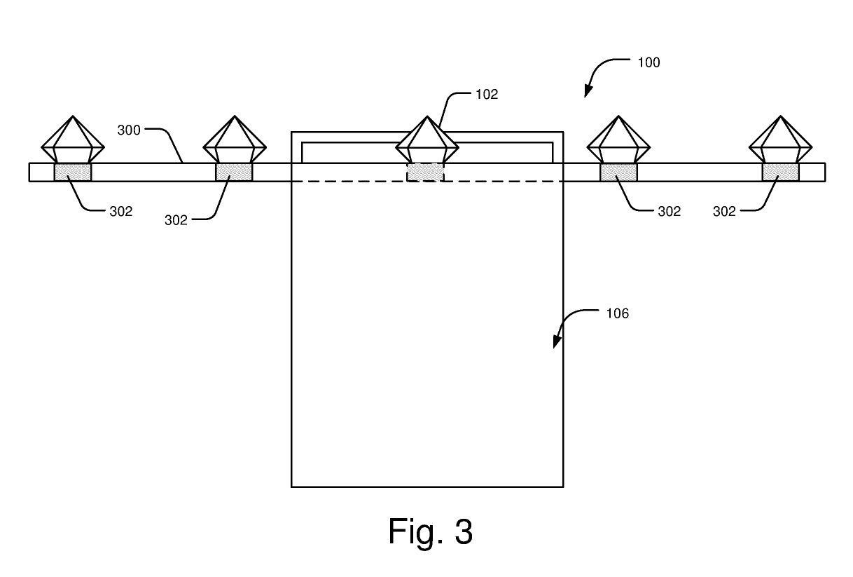

In addition, the system 100 can be adapted to achieve successive identification of a plurality of gemstones. FIG. 3 and FIG. 4 illustrate embodiments of the system 100 adapted for the above mentioned purpose. For example, as shown in FIG. 3, the system 100 can include a conveyor belt 300 having a plurality of gemstone holders 302 provided thereon and each gemstone holder 302 can be used for grasping the gemstone 102. The conveyor belt 300 can be provided with a drive through an actuator (not shown), in turn driven by a processing unit (not shown), to regulate the positioning of the gemstones 102 for capturing the pattern.

Further, in the embodiment shown in FIG. 4, the system 100 can include a multi-holder plate 400 having a plurality of gemstone holders 402 and an actuator assembly 404 coupled to the multi-holder plate 400 and/or the optoelectronic assembly 106 for achieving a relative motion between the gemstones 102 and the optoelectronic assembly 106 for positioning the optoelectronic assembly 106 with respect to the gemstone 102. In another case, an actuator assembly 404 can be individually coupled to each gemstone holder 402 on the multi-holder plate 400 to individually regulate the movement of the gemstone holder 402 with respect to the optoelectronic assembly 106.

In addition, the present subject matter relates to the method for mobile gemstone identification. In an implementation, the method can include securing a gemstone in a gemstone holder 104, 202, 402, illuminating the gemstone 102, and capturing the pattern indicative of the optical response of the gemstone 102 formed on a screen 110, in response to the radiations being incident on the gemstone 102. As explained above, the screen 110 is formed as having a plurality of regions, at least one region having greater mass than the other regions and the pattern of the gemstone 102 being formed on the region having greater mass.

Although implementations for methods and systems for mobile identification of gemstones are described, it is to be understood that the present subject matter is not necessarily limited to the specific features or methods described. Rather, the specific features and methods are disclosed as implementations for identification of an activity performed by a subject based on sensor data analysis.

I/We claim:

1. A mobile gemstone identification system (100) comprising:
a gemstone holder (104, 202, 302, 402) to hold a gemstone (102);
an optoelectronic assembly (106) comprising,
an illumination device (108) to illuminate the gemstone (102), wherein radiations are incident on the gemstone (102) by the illumination device (108); and
a screen (110) to form a pattern indicative of an optical response of the gemstone (102), in response to the radiations incident on the gemstone (102), wherein the screen (110) is formed as having a plurality of regions (114, 116) having different masses, and the pattern is to be formed substantially on a region (114) having greater mass than other regions (116).

2. The mobile gemstone identification system (100) as claimed in claim 1, wherein the region (114) has a darker shade than the other regions (116) on a surface of the screen (110) facing the gemstone (102).

3. The mobile gemstone identification system (100) as claimed in claim 1, wherein the plurality of regions (114, 116) have different cross-sectional thicknesses.

4. The mobile gemstone identification system (100) as claimed in claim 1, wherein the screen (110) is formed as having a layered structure.

5. The mobile gemstone identification system (100) as claimed in claim 1, wherein the screen (110) has a curvature at least on a surface facing the gemstone (102).

6. The mobile gemstone identification system (100) as claimed in claim 5, wherein the screen (110) has one of a biconvex cross section, a plano-convex cross section, biconcave cross section, and a plano-concave cross section.

7. The mobile gemstone identification system as claimed in claim 1, wherein the optoelectronic assembly (106) comprises an optical device (120) to direct the radiations from the illumination device (108) onto the gemstone (102), wherein the gemstone (102) is non-linearly positioned with respect to the illumination device (108).

8. The mobile gemstone identification system (100) as claimed in claim 1, wherein the gemstone holder (302) is provided on a conveyor belt (300), the conveyor belt (300) having a plurality of gemstone holders (302) provided thereon.

9. The mobile gemstone identification system (100) as claimed in claim 1, wherein the optoelectronic assembly (106) comprises an image capturing device (118) to capture the pattern of the gemstone (102) to be formed on the screen (110).

10. The mobile gemstone identification system (100) as claimed in claim 1 comprising an actuator assembly (404) coupled to at least one of the gemstone holder (104, 202, 302, 402) and the optoelectronic assembly (106), to achieve a relative motion between the gemstone holder (104, 202, 302, 402) and the optoelectronic assembly (106) to position the optoelectronic assembly (106) with respect to the gemstone holder (104, 202, 302, 402).

11. The mobile gemstone identification system (100) as claimed in claim 1, wherein the mobile gemstone identification system (100) is to couple to at least one global database to at least one of verify the gemstone (102) and updating the pattern of the gemstone (102) in the global database, the global database being a central repository having a plurality of certificates for authenticate gemstones stored therein.

12. A method for mobile gemstone identification, the method comprising:
securing a gemstone (102) in a gemstone holder (104, 202, 302, 402);
illuminating the gemstone (102), wherein radiations are incident on the gemstone (102); and
capturing a pattern indicative of an optical response of the gemstone (102) formed on a screen (110), in response to the radiations being incident on the gemstone (102), the screen (110) being formed as having a plurality of regions (114, 116) having different masses, wherein the pattern is to be formed substantially on a region (114) having greater mass than other regions (116).
